(19) United States
(12) Patent Application Publication
(10) Pub. No.: US 2018/0072985 A1
(43) Pub. Date: Mar. 15, 2018

(54) GLUCONACETOBACTER HAVING ENHANCED CELLULOSE PRODUCTIVITY

(71) Applicant: Samsung Electronics Co., Ltd., Suwon-si (KR)

(72) Inventors: Soonchun Chung, Seoul (KR); Jiae Yun, Hwaseong-si (KR); Jinkyu Kang, Hwaseong-si (KR); Jinhwan Park, Suwon-si (KR)

(21) Appl. No.: 15/702,267

(22) Filed: Sep. 12, 2017

(30) Foreign Application Priority Data
Sep. 12, 2016 (KR) 10-2016-0117368

(51) Int. Cl. C12N 1/20; C12N 9/88; C12P 19/04; C12N 9/12

(52) U.S. Cl. CPC C12N 1/20; C12N 9/88; C12Y 207/07009; C12P 19/04; C12N 9/1241; C12Y 401/02013

(57) ABSTRACT

A microorganism of the genus Gluconacetobacter has enhanced cellulose productivity due to overexpression of fructose-bisphosphate aldolase, and optionally, phosphoglucomutase, UTP-glucose-1-phosphate uridylyltransferase, or cellulose synthase. A method of producing cellulose and a method of producing the microorganism are provided.

### CROSS-REFERENCE TO RELATED APPLICATION

This application claims the benefit of Korean Patent Application No. 10-2016-0117368, filed on Sep. 12, 2016, in the Korean Intellectual Property Office, the entire disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: One 29,343 byte ASCII (Text) file named “727699_ST25.TXT,” created Sep. 11, 2017.

### BACKGROUND

### 1. Field

The present disclosure relates to a microorganism of the genus Gluconacetobacter having enhanced cellulose productivity, a method of producing cellulose using the same, and a method of producing the microorganism.

### 2. Description of the Related Art

Cellulose can be harvested from cultures of a microorganism. The so-produced cellulose, is primarily composed of glucose in the form of β-1,4 glucan units. On a larger scale, cellulose molecules form a network structure of fibril bundles. This cellulose is also called ‘bio-cellulose or microbial cellulose’.

Unlike plant cellulose, microbial cellulose is pure cellulose entirely free of lignin or hemicellulose. Microbial cellulose, which is typically 100 nm or less in width, has network structure of bundles of cellulose nanofibers, and has characteristic properties such as high water absorption and retention capacity, high tensile strength, high elasticity, and high heat resistance, compared to plant cellulose. They make it suitable for use in a variety of fields, including cosmetics, medical products, dietary fibers, audio speaker diaphragms, functional films, etc.

Acetobacter, Agrobacteria, Rhizobia, and Sarcina have been reported as microbial cellulose-producing strains. Upon static culture under aerobic conditions, cellulose with a three-dimensional network structure is formed as a thin film on the surface of a culture of these microorganisms.

Still, there is a demand for a recombinant microorganism of the genus Gluconacetobacter having enhanced cellulose productivity.

### SUMMARY

An aspect provides a microorganism of the genus Gluconacetobacter having enhanced cellulose productivity. The recombinant microorganism comprises a genetic modification that increases fructose-bisphosphate aldolase (FBA) activity.

Another aspect provides a method of producing cellulose using the microorganism. The method comprises culturing a recombinant microorganism of the genus Gluconacetobacter having enhanced cellulose productivity in a culture medium and collecting cellulose from the culture medium, wherein the microorganism comprising a genetic modification that increases fructose-bisphosphate aldolase activity.

Also provided is a method of producing the recombinant microorganism having enhanced cellulose productivity, the method comprising introducing a genetic modification that increases fructose-bisphosphate aldolase (FBA) activity into a microorganism of the genus Gluconacetobacter.

### DETAILED DESCRIPTION

The term “parent cell” refers to a cell in a state immediately prior to a particular genetic modification, for example, a cell that serves as a starting material for producing a cell having a genetic modification that increases or decreases the activity of one or more proteins. A parent cell, thus, is a cell without a particular referenced genetic modification, but with the other genotypic and phenotypic traits of the genetically modified cell. Although the “parent cell” does not have the specific referenced genetic modification, the parent cell may be engineered in other respects and, thus, might not be a “wild-type” cell (though it may also be a wild-type cell if no other modifications are present). Thus, the parent cell may be a cell used as a starting material to produce a genetically engineered microorganism having an inactivated or decreased activity of a given protein (e.g., a protein having a sequence identity of about 95% or more to a protein having a sequence identity of about 95% or more with respect to fructose-bisphosphate aldolase or other protein) or a genetically engineered microorganism having an increased activity of a given protein (e.g., a protein having a sequence identity of about 95% or more to the protein). By way of further illustration, with respect to a cell in which a gene encoding a protein has been modified to reduce gene activity, the parent cell may be a microorganism including an unaltered, “wild-type” gene. The same comparison is applied to other genetic modifications.

The term “increase in activity” or “increased activity”, as used herein, refers to a detectable increase in an activity of a cell, a protein, or an enzyme including a modified (e.g., genetically engineered) cell, protein, or enzyme that is higher than that of a comparative (e.g., parent, wild-type, or control) cell, protein, or enzyme of the same type, such as a cell, protein, or enzyme that does not have a given genetic modification (e.g., original or “wild-type” cell, protein, or enzyme). “Cell activity” refers to an activity of a particular protein or enzyme of a cell. For example, an activity of a modified or engineered cell, protein, or enzyme can be increased by about 5% or more, about 10% or more, about 15% or more, about 20% or more, about 30% or more, about 50% or more, about 60% or more, about 70% or more, or about 100% or more than an activity of a non-engineered cell, protein, or enzyme of the same type, i.e., a wild-type cell, protein, or enzyme. A cell having an increased activity of a protein or an enzyme can be identified by using any method known in the art.

An increase in activity of an enzyme or a polypeptide can be achieved by an increase in the expression or specific activity thereof. The increase in the expression can be achieved by introduction of an exogenous gene encoding the enzyme or the polypeptide into a cell, by an increase in a copy number in a gene encoding the enzyme or polypeptide in a cell, or by a mutation (including point mutations and promoter-swaps) in the regulatory region of an endogenous polynucleotide. The microorganism receiving the exogenous gene may already contain a copy of the gene endogenously, or might not include the gene prior to its introduction. The gene may be operably linked to a regulatory sequence that allows expression thereof, for example, a promoter, an enhancer, a polyadenylation region, or a combination thereof (e.g., by inclusion in an expression cassette of an appropriate vector). An exogenous gene refers to a gene introduced into a cell from the outside. The introduced exogenous gene may be endogenous or heterologous with respect to the host cell. An endogenous gene refers to a gene that already exists in the genetic material of a microorganism (e.g., a native gene). A heterologous gene is one that does not normally exist in the genetic material of a given microorganism (e.g., foreign or not native).

An increase in the copy number can be caused by introduction of an exogenous gene or by amplification of an endogenous gene, and encompasses genetically engineering a cell so that the cell has a gene that does not exist in a non-engineered cell (i.e., introduction of an exogenous heterologous gene, thereby increasing copy number from 0 to 1). The introduction of the gene can be mediated by a vehicle such as a vector. The introduction can result in a gene that is not integrated into a genome (e.g. a transient or stable episome such as a plasmid or artificial chromosome), or an integration of the gene into the genome (e.g., by homologous recombination). The introduction can be performed, for example, by introducing a vector into the cell, the vector including a polynucleotide encoding a target polypeptide, and then replicating the vector in the cell or by integrating the polynucleotide into the genome.

The introduction of the gene may be performed by a known method, such as transformation, transfection, and electroporation. The gene may be introduced via a vehicle or by itself. As used herein, the term “vehicle” refers to a nucleic acid molecule capable of delivering other nucleic acids linked thereto (e.g., a vector, a nucleic acid construct, or a cassette). Examples of a vector include a plasmid vector, a virus-derived vector, etc. A plasmid (e.g., plasmid expression vector) is a circular double-stranded DNA molecule linkable with other DNA. Examples of viral expression vectors include replication-defective retrovirus, adenovirus, adeno-associated virus, and the like.

The term “gene”, as used herein, refers to a nucleic acid fragment expressing a specific protein, and the fragment may or may not include one or more regulatory sequences (e.g., 5′-non coding sequence and/or 3′-non coding sequence).

“Sequence identity” of a nucleic acid or a polypeptide, as used herein, refers to the extent of identity between nucleotides or amino acid residues of sequences obtained after the sequences are aligned so as to best match in certain comparable regions. Sequence identity is a value obtained by comparing two sequences in certain comparable regions via optimal alignment of the two sequences, in which portions of the sequences in the certain comparable regions may be added or deleted compared to reference sequences. A percentage of sequence identity may be calculated by, for example, comparing two optimally aligned sequences in the entire comparable regions, determining the number of locations in which the same amino acids or nucleic acids appear to obtain the number of matching locations, dividing the number of matching locations by the total number of locations in the comparable regions (e.g., the size of a range), and multiplying a result of the division by 100 to obtain the percentage of the sequence identity. The percentage of the sequence identity may be determined using a known sequence comparison program, for example, BLASTN (NCBI), BLASTP (NCBI), CLC Main Workbench (CLC bio), or MegAlign™ (DNASTAR Inc), or Needleman-Wunsch global alignment algorithm (e.g., EMBOSS Needle). Unless otherwise specified, selection of parameters used for operating the program is as follows: Ktuple=2, Gap Penalty=4, and Gap length penalty=12. Sequence identity comparisons can also be performed manually, where optimal alignment is clearly ascertained.

Various levels of sequence identity may be used to identify various types of polypeptides or polynucleotides having the same or similar functions or activities. For example, the sequence identity may include a sequence identity of about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100%.

Where a polynucleotide sequence encoding a given protein, other polynucleotide sequences can be substituted due to the degeneracy of the genetic code.

The term “genetic modification”, as used herein, includes an artificial alteration in a constitution or structure of genetic material of a cell, which can be accomplished using any suitable technique.

In the present invention, unless otherwise specified, % represents w/w %.

An aspect provides a recombinant microorganism of the genus Gluconacetobacter having enhanced cellulose productivity, the microorganism including a genetic modification that increases the activity of fructose-bisphosphate aldolase (FBA).

Fructose-bisphosphate aldolase (FBA) catalyzes the reaction: fructose-1,6-bisphosphate (FBP) [symbol] dihydroxyacetone (DHAP)+glyceraldehyde 3-phosphate (G3P). The fructose-bisphosphate aldolase can belong to EC 4.1.2.13. The fructose-bisphosphate aldolase can be exogenous or endogenous. The fructose-bisphosphate aldolase can be in the form of a monomer consisting of a single polypeptide. The fructose-bisphosphate aldolase can be selected from the group consisting of fructose-bisphosphate aldolases derived from the genus Gluconacetobacter, the genus Bacillus, the genus Mycobacterium, the genus Zymomonas, the genus Vibrio, and the genus Escherichia.

The fructose-bisphosphate aldolase can be a polypeptide having a sequence identity of about 95% or more with respect to an amino acid sequence of SEQ ID NO: 1.

In the microorganism, the genetic modification can increase expression of a gene encoding fructose-bisphosphate aldolase. For instance, the genetic modification can be an increase in the copy number of the fructose-bisphosphate aldolase gene (e.g., a gene encoding the polypeptide having a sequence identity of about 95% or more with respect to the amino acid sequence of SEQ ID NO: 1). For example, the gene can have a nucleotide sequence of SEQ ID NO: 2, or a sequence having at least 85%, 90%, or 95% sequence identity to the sequence of SEQ ID NO: 2.

In one aspect, the genetic modification may be the introduction of a gene encoding fructose-bisphosphate aldolase, for example, via a vehicle such as a vector. The fructose-bisphosphate aldolase and gene encoding same that is introduced into the microorganism may be endogenous or heterologous. The gene encoding fructose -bisphosphate aldolase once introduced may integrate within the host genome or remain independent (outside) of the chromosome. Furthermore, the genetic modification may include the introduction of a plurality of genes encoding fructose-bisphosphate aldolase, for example, 2 or more, 5 or more, 10 or more, 50 or more, 100 or more, or 1000 or more genes, which may be the same (e.g., multiple copies of a gene) or different provided they encode fructose bisphosphate aldolase.

The microorganism can be of the genus Gluconacetobacter, for example, G. aggeris, G. asukensis, G. azotocaptans, G. diazotrophicus, G. entanii, G. europaeus, G. hansenii, G. intermedius, G. johannae, G. kakiaceti, G. kombuchae, G. liquefaciens, G. maltaceti, G. medellinensis, G. nataicola, G. oboediens, G. rhaeticus, G. sacchari, G. saccharivorans, G. sucrofermentans, G. swingsii, G. takamatsuzukensis, G. tumulicola, G. tumulisoli, or G. xylinus (also called “Komagataeibacter xylinus”).

The microorganism can further include one or more genetic modifications selected from the group consisting of a genetic modification that increases activity of phosphoglucomutase (PGM) which catalyzes conversion of glucose-6-phosphate to glucose-1-phosphate, a genetic modification that increases activity of UTP-glucose-1-phosphate uridylyltransferase (UPG) which catalyzes conversion of glucose-1-phosphate to UDP-glucose, and a genetic modification that increases activity of cellulose synthase (CS) which catalyzes conversion of UDP-glucose to cellulose. The genetic modification may be an increase in the copy number of the above one or more genes. PGM may belong to EC 2.7.7.9, and UGP may belong to EC 5.4.2.2 or EC 5.4.2.5.

The polypeptide having PGM activity can be, for instance, a polypeptide having a sequence identity of about 95% or more with respect to an amino acid sequence of SEQ ID NO: 4. Similarly, the polypeptide having UPG activity can be a polypeptide having a sequence identity of about 95% or more with respect to an amino acid sequence of SEQ ID NO: 6, and the polypeptide having CS activity can be a polypeptide having a sequence identity of about 95% or more with respect to an amino acid sequence of SEQ ID NO: 19.

In an embodiment, the microorganism may have an increase in the copy number of one or more genes selected from the group consisting of a gene having a nucleotide sequence of SEQ ID NO: 3 (or a sequence having at least 85%, 90%, or 95% sequence identity to the sequence of SEQ ID NO: 3), a gene having a nucleotide sequence of SEQ ID NO: 5 (or a sequence having at least 85%, 90%, or 95% sequence identity to the sequence of SEQ ID NO: 5), and a gene having a nucleotide sequence of SEQ ID NO: 18 (or a sequence having at least 85%, 90%, or 95% sequence identity to the sequence of SEQ ID NO: 18).

Another aspect provides a method of producing cellulose. The method includes culturing the recombinant microorganism of the genus Gluconacetobacter having enhanced cellulose productivity. The microorganism includes a genetic modification that increases the activity of fructose-bisphosphate aldolase. The microorganism is cultured in a medium to produce cellulose; and the cellulose is obtained (isolated or otherwise collected) from the culture.

The culturing may be performed in a medium containing a suitable carbon source, for example, glucose. The medium used for culturing the microorganism may be any general medium suitable for host cell growth, such as a minimal or complex medium containing appropriate supplements. The suitable medium may be commercially available or prepared by a known preparation method.

The medium may be a medium that may satisfy the requirements of the particular microorganism used. The medium may be a medium including components selected from the group consisting of a carbon source, a nitrogen source, a salt, trace elements, and combinations thereof. The medium may include ethanol of about 0.5% to about 3% (v/v), for example, about 0.5% to about 2.5% (v/v), about 0.75% to about 2.25% (v/v), or about 1.0% to about 2.0% (v/v).

The culturing conditions may be appropriately controlled for the production of cellulose. The culturing may be performed under aerobic conditions for cell proliferation. The culturing may be performed by static culture without shaking. The culturing may be performed with a low density of the microorganism. The density of the microorganism may be a density which provides intercellular space sufficient to not to disturb secretion of cellulose.

The term “culture conditions”, as used herein, mean conditions for culturing the microorganism. Such culture conditions may include, for example, a carbon source, a nitrogen source, or an oxygen condition utilized by the microorganism. The carbon source that may be utilized by the microorganism may include monosaccharides, disaccharides, or polysaccharides. The carbon source may include glucose, fructose, mannose, or galactose as an assimilable sugar. The nitrogen source may be an organic nitrogen compound or an inorganic nitrogen compound. The nitrogen source may be exemplified by amino acids, amides, amines, nitrates, or ammonium salts. An oxygen condition for culturing the microorganism may be an aerobic condition of a normal oxygen partial pressure, or a low-oxygen condition including about 0.1% to about 10% of oxygen in the atmosphere. A metabolic pathway may be modified in accordance with a carbon source or a nitrogen source that may be actually used by a microorganism.

The method may include separating and collecting the cellulose from the culture. Separation may be accomplished, for example, by collecting a cellulose pellicle formed on the top of the medium. The cellulose pellicle may be collected by physically stripping off the cellulose pellicle or by removing the medium. The separation may involve collecting the cellulose pellicle while maintaining its shape without damage.

Still another aspect provides a method of producing the microorganism having enhanced cellulose productivity. The method includes introducing a genetic modification that increases fructose bisphosphate aldolase (FBA) activity into the microorganism of the genus Gluconacetobacter. The genetic modification may be any described herein with respect to the recombinant microorganism. Thus, for instance, the genetic modification can be, for instance, introduction of an exogenous gene encoding fructose bisphosphate aldolase into the microorganism of the genus Gluconacetobacter. The gene may be heterologous or endogenous. The introducing of the gene encoding fructose-bisphosphate aldolase can be achieved by introducing a vehicle including the gene into the microorganism. In addition or instead, the genetic modification can include amplification of an endogenous gene, manipulation of the regulatory sequence of the gene, or manipulation of the sequence of the gene itself, such as by insertion, substitution, conversion, or addition of nucleotides.

The method may further include introducing one or more genetic modifications selected from the group consisting of a genetic modification that increases activity of PGM that catalyzes conversion of glucose-6-phosphate to glucose-1-phosphate, a genetic modification that increases activity of UPG that catalyzes conversion of glucose -1-phosphate to UDP-glucose, and a genetic modification that increases activity of cellulose synthase that catalyzes conversion of UDP-glucose to cellulose, as previously described herein. For instance, the genetic modification may increase the copy number of one or more genes selected from the group consisting of a gene encoding a polypeptide having PGM activity, the polypeptide having a sequence identity of about 95% or more with respect to the amino acid sequence of SEQ ID NO: 4 and a gene encoding a polypeptide having UPG activity, the polypeptide having a sequence identity of about 95% or more with respect to the amino acid sequence of SEQ ID NO: 6. The introducing of the genetic modification may be achieved by increasing the copy number of one or more genes selected from the group consisting of a gene having a nucleotide sequence of SEQ ID NO: 3 and a gene having a nucleotide sequence of SEQ ID NO: 5.

The recombinant microorganism of the genus Gluconacetobacter having enhanced cellulose productivity may be used to produce cellulose efficiently and/or in a high yield.

[symbol] .

Reference will now be made in detail to embodiments. In this regard, the present embodiments may have different forms and should not be construed as being limited to the descriptions set forth herein. As used herein, the term “and/or” includes any and all combinations of one or more of the associated listed items.

In accordance with an embodiment, a method of producing the recombinant organism with enhanced cellulose efficiency comprises making the genetic modifications described above or in the Examples below.

### Example 1

### Preparation of K. xylinus Including Fructose-bisphosphate Aldolase Gene and Production of Cellulose

In this Example, K. xylinus DSM2325 was transformed with an exogenous or endogenous fructose-bisphosphate aldolase (FBA) gene. The resulting microorganism cultured to produce cellulose to examine the effects of the gene introduction on cellulose productivity.

1. Introduction of Fructose-bisphosphate Aldolase Gene

A K. xylinus DSM2325 GX_1979 gene with a nucleotide sequence of SEQ ID NO: 2 was introduced into a K. xylinus DSM2325 M9 strain. The specific introduction procedures performed are as follows.

(1) Construction of Vector

PCR was performed using a pTSa-EX1 vector (SEQ ID NO: 11) as a template and a set of primers of either F1-F (SEQ ID NO: 7) and F1-R (SEQ ID NO: 8) or F2-F (SEQ ID NO: 9) and F2-R (SEQ ID NO: 10) to obtain a PCR product of 1.9 kb or 0.3 kb, respectively. The PCR product had a point mutation in the sequence of the vector, and one restriction enzyme site was removed therefrom. This PCR product was cloned into BamHI/SaII restriction sites of the pTSa-EX1 vector using an In-Fusion GD cloning kit (Takara) to prepare a pTSa-EX11 vector.

Next, an open reading frame (ORF) of a fructose-bisphosphate aldolase GX_1979 gene was obtained by gel extraction after PCR using genomic DNA of the K. xylinus DSM2325 M9 strain as a template and a set of primers of SEQ ID NO: 12 and SEQ ID NO: 13.

The ORF of the gene was cloned into a SaII restriction site of the pTSa-EX11 vector using an In-Fusion GD cloning kit (Takara) to prepare an overexpression vector pTSa-GX1979.

(2) Transformation

The K. xylinus DSM2325 M9 strain was spread on a plate containing an HS-agar medium supplemented with 2% glucose, and cultured at 30° C. for 3 days. The strain thus cultured was flushed with 2 ml of sterile water, and colonies were pooled. The colonies were transferred to a 50 ml falcon tube, followed by vortexing for 2 minutes. The 2% glucose-containing HS-agar medium included 0.5% peptone, 0.5% yeast extract, 0.27% Na₂HPO₄, 0.15% citric acid, 2% glucose, and 1.5% bacter-agar. Thereafter, 1% cellulase (sigma, Cellulase from Trichoderma reesei ATCC 26921) was added and allowed to react at 30° C. and 160 rpm for 2 hours. Then, the colonies were washed with 1 mM HEPES buffer-containing medium and then washed with 15 (w/w) % glycerol three times, followed by resuspension in 1 ml of 15 (w/w) % glycerol.

100 μl of competent cells thus prepared were transferred to a 2-mm electro-cuvette, and then 3 μg of pTSa-GX1979 plasmid was added thereto, followed by transformation via electroporation (2.4 kV, 200Ω, 25 μF). The transformed cells were resuspended in 1 ml of HS medium containing 2% glucose, and then transferred to a 14-ml round-tube, followed by incubation at 30° C. and 160 rpm for 2 hours. Then, the cells were spread on a plate containing an HS-agar medium supplemented with 2% glucose, 1 (v/v) % ethanol, and 5 μg/ml tetracycline, and cultured at 30° C. for 5 days.

(3) Test of Glucose Consumption and Cellulose Production

The strain cultured in (2) was inoculated into a 250-mL flask containing 25 ml of HS medium supplemented with 5% glucose, 1% ethanol, and 5 μg/ml tetracycline, and cultured at 30° C. and 230 rpm for 5 days. As a result, cellulose (hereinafter, also referred to as “cellulose nanofiber (CNF)”) was formed on the surface where the medium was in contact with air. CNF thus produced was collected as a pellicle, washed with 0.1 N NaOH and distilled water at 60° C., and then freeze-dried to remove H2O, followed by weighing.

Glucose and gluconate were analyzed by HPLC equipped with an Aminex HPX -87H column (Bio-Rad, USA). Table 1 shows CNF production and yield, gluconate yield, and glucose consumption of the K. xylinus strain introduced with an Fba gene.

TABLE 1

| | CNF | | Gluconate | |
|---|---|---|---|---|
| | production | CNF yield | yield | Glucose |
| Strain | (g/L) | (g/g) | (g/g) | consumption (g/L) |
| pTSa-EX1 | 1.36 | 0.07 | 1.16 | 18.9 |
| pTSa-GX1979 | 1.96 | 0.07 | 0.99 | 27.1 |

In Table 1, pTSa-EX1 represents a pTSa-EX1 vector-containing K. xylinus strain used as a control group, and pTSa-GX1979 represents a pTSa-GX1979 vector-containing K. xylinus strain used as an experimental group. As shown in Table 1, the K. xylinus strain (pTSa-GX1979) transformed with the FBA GX_1979 gene showed a 43.6% increase in CNF production, as compared with the pTSa-EX1 control strain. In Table 1, CNF yield and gluconate yield represents grams of CNF/gram of glucose and grams of gluconate/gram of glucose, respectively. As shown in Table 1, the experimental group and the control group showed similar CNF yields. The experimental group showed a 25% decrease in gluconate yield, as compared with the control group. Further, the experimental group showed a 43.0% increase in glucose consumption, as compared with the control group. The glucose consumption represents glucose g/medium 1 L.

### Example 2

### Introduction of Phosphoglucomutase Gene or UTP-glucose-1-phosphate Uridylyltransferase Gene

Glucose-6-phosphate produced via gluconeogenesis is converted to UTP -glucose which is a substrate of cellulose synthase by phosphoglucomutase (hereinafter, also referred to as “PGM”) and UTP-glucose-1-phosphate uridylyltransferase (hereinafter, also referred to as “UGP”) enzymes. In this Example, K. xylinus was introduced with a K. xylinus DSM2325 M9 strain-derived PGM gene or UGP gene, and effects of the gene introduction on cellulose productivity were examined.

(1) Construction of Vector

Open reading frames of the PGM GX_1215 gene (SEQ ID NO: 3) and the UGP GX_2556 gene (SEQ ID NO: 5) were obtained by gel extraction after PCR using genomic DNA of the K. xylinus DSM2325 M9 strain as a template and a set of primers of either SEQ ID NOS: 14 and 15 or SEQ ID NOS: 16 and 17. The obtained ORF of each gene was cloned into a SaII restriction site of a pTSa-EX11 vector using an In-Fusion GD cloning kit (Takara) to prepare an overexpression vector. Hereinafter, these vectors are referred to as a pTSa-GX1215 vector and a pTSa-GX2556 vector, respectively.

(2) Transformation

K. xylinus DSM2325 M9 strain was spread on a plate containing an HS-agar medium supplemented with 2% glucose, and cultured at 30° C. for 3 days. The strain thus cultured was flushed with 2 ml of sterile water, and colonies were pooled. The colonies were transferred to a 50 ml falcon tube, followed by vortexing for 2 minutes. The 2% glucose-containing HS-agar medium included 0.5% peptone, 0.5% yeast extract, 0.27% Na₂HPO₄, 0.15% citric acid, 2% glucose, and 1.5% bacter-agar. Thereafter, 1% cellulase (sigma, Cellulase from Trichoderma reesei ATCC 26921) was added and allowed to react at 30° C. and 160 rpm for 2 hours. Then, the colonies were washed with 1 mM HEPES buffer-containing medium and then washed with 15 (w/w) % glycerol three times, followed by resuspension in 1 ml of 15 (w/w) % glycerol.

100 μl of competent cells thus prepared were transferred to a 2-mm electro-cuvette, and then 3 μg of pTSa-GX1215 or pTSa-GX2556 plasmid was added thereto, followed by transformation via electroporation (2.4 kV, 200Ω, 25 μF). The transformed cells were resuspended in 1 ml of HS medium containing 2% glucose, and then transferred to a 14-ml round-tube, followed by incubation at 30° C. and 160 rpm for 2 hours. Then, the cells were spread on a plate containing an HS-agar medium supplemented with 2% glucose, 1 (v/w) % ethanol, and 5 μg/ml tetracycline, and cultured at 30° C. for 5 days.

(3) Test of Glucose Consumption and Cellulose and Gluconate Productions

The strain cultured in (2) was inoculated into a 250-mL flask containing 25 ml of HS medium supplemented with 5% glucose, 1% ethanol, and 5 μg/ml tetracycline, and cultured at 30° C. and 230 rpm for 5 days. As a result, cellulose (hereinafter, also referred to as “cellulose nanofiber (CNF)”) was formed on the surface where the medium was in contact with air. CNF thus produced was harvested as the pellicle and washed with 0.1 N NaOH and distilled water at 60° C., then freeze-dried to remove H₂O, and then weighed.

Glucose and gluconate were analyzed by HPLC. Table 2 shows CNF production, gluconate production, and glucose consumption of the K. xylinus strains transformed with either the PGM gene or UGP gene.

TABLE 2

| | CNF | Gluconate | Glucose | CNF | |
|---|---|---|---|---|---|
| | production | production | consumption | yield | Gluconate |
| Strain | (g/L) | (g/L) | (g/L) | (g/g) | yield (g/g) |
| pTSa-EX1 | 1.61 | 25.14 | 26.83 | 0.06 | 0.94 |
| pTSa-GX1215 | 1.82 | 25.25 | 26.39 | 0.07 | 0.96 |
| pTSa-GX2556 | 2.10 | 29.22 | 34.47 | 0.06 | 0.85 |

In Table 4, pTSa-EX1 represents a control strain of a pTSa-EX1 vector-containing K. xylinus strain, pTSa-GX1215 represents an experimental PGM -transformed strain of a pTSa-GX1215-containing K. xylinus strain, and pTSa-GX2556 represents an experimental UGP-transformed strain of a pTSa-GX2556 vector -containing K. xylinus strain. As shown in Table 2, the PGM gene- and UGP gene -introduced strains respectively showed increases of 13.0% and 30.4% in CNF production, as compared with the control group, respectively.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described, it will be understood by those of ordinary skill in the art that various changes in form and details may be made therein without departing from the spirit and scope as defined by the following claims.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant microorganism of the genus Gluconacetobacter having enhanced cellulose productivity, the recombinant microorganism comprising a genetic modification that increases fructose-bisphosphate aldolase (FBA) activity.

2. The microorganism of claim 1, wherein the genetic modification is to increase the copy number of a gene encoding the fructose-bisphosphate aldolase.

3. The microorganism of claim 2, comprises an exogenous gene encoding the fructose-bisphosphate aldolase.

4. The microorganism of claim 2, wherein the fructose-bisphosphate aldolase belongs to EC 4.1.2.13.

5. The microorganism of claim 1, wherein the fructose-bisphosphate aldolase is a polypeptide having a sequence identity of 95% or more with respect to an amino acid sequence of SEQ ID NO: 1.

6. The microorganism of claim 2, wherein the gene has a nucleotide sequence of SEQ ID NO: 2.

7. The microorganism of claim 1, wherein the microorganism is Gluconacetobacter xylinus.

8. The microorganism of claim 1, further comprising one or more genetic modifications selected from the group consisting of a genetic modification that increases the activity of phosphoglucomutase (PGM), which catalyzes conversion of glucose-6-phosphate to glucose-1-phosphate; a genetic modification that increases the activity of UTP-glucose-1-phosphate uridylyltransferase (UPG), which catalyzes conversion of glucose-1-phosphate to UDP-glucose; and a genetic modification that increases the activity of cellulose synthase (CS), which catalyzes conversion of UDP-glucose to cellulose.

9. The microorganism of claim 8, wherein the microorganism has an increase in the copy number of one or more genes selected from the group consisting of a gene encoding phosphoglucomutase, which catalyzes conversion of glucose-6-phosphate to glucose-1-phosphate; a gene encoding UTP-glucose-1-phosphate uridylyltransferase, which catalyzes conversion of glucose-1-phosphate to UDP -glucose; and a gene encoding cellulose synthase, which catalyzes conversion of UDP -glucose to cellulose.

10. The microorganism of claim 8, wherein the microorganism has an increase in the copy number of one or more of a gene encoding a phosphoglucomutase having about 95% or more sequence identity to SEQ ID NO: 4; or a gene encoding a UTP-glucose-1-phosphate uridylyltransferase having about 95% or more sequence identity to SEQ ID NO: 6.

11. The microorganism of claim 8, wherein the microorganism has an increase in the copy number of one or more of a gene having a nucleotide sequence of SEQ ID NO: 3 or a gene having a nucleotide sequence of SEQ ID NO: 5.

12. A method of producing cellulose, the method comprising:
culturing the recombinant microorganism of claim 1 and collecting cellulose from the culture .

13. The method of claim 12, wherein the recombinant microorganism comprises an increase in the copy number of a gene encoding a fructose-bisphosphate aldolase.

14. The method of claim 12, comprises an exogenous gene encoding the fructose-bisphosphate aldolase.

15. The method of claim 12, wherein the fructose-bisphosphate aldolase belongs to EC 4.1.2.13.

16. The method of claim 12, wherein the fructose-bisphosphate aldolase has 95% or more sequence identity to SEQ ID NO: 1.

17. The method of claim 13, wherein the gene encoding the fructose -bisphosphate aldolase has a nucleotide sequence of SEQ ID NO: 2.

18. The method of claim 13, wherein the recombinant microorganism is G. xylinus.

19. The method of claim 13, wherein the microorganism comprises one or more genetic modifications selected from the group consisting of a genetic modification that increases the activity of phosphoglucomutase, which catalyzes conversion of glucose-6-phosphate to glucose-1-phosphate; a genetic modification that increases activity of UTP-glucose-1-phosphate uridylyltransferase, which catalyzes conversion of glucose-1-phosphate to UDP-glucose; and a genetic modification that increases activity of cellulose synthase, which catalyzes conversion of UDP-glucose to cellulose.

20. A method of producing the microorganism of claim 1, the method comprising introducing a genetic modification that increases fructose-bisphosphate aldolase (FBA) activity into a microorganism of the genus Gluconacetobacter.


SEQUENCE LISTING (19 sequences)

SEQ ID NO: 1 - protein, 362 aa, Gluconacetobacter xylinus
  misc_feature (1)..(362): DSM2325 M9
MAHSARPSLPPGVVTGENYRKLVATCCSEGYALPAVNVVGTDSINAVLEAAARNRSDVIIQMSNGGARFYAGEGMKDQHRARVLGAVAAARHVHTLAAAYGVCVILHTDHADRKLLPWVSDLIDESEAAVHATGQPLFSSHMIDLSAEPLDDNIAECARFLRRMAPLGIGLEIELGVTGGEEDGIGHDLDDGADNAHLYTQPADVLRAYNELSPLGFVTIAASFGNVHGVYAPGNVKLRPEILLHSQQAVSEATGQGERPLALVFHGGSGSEQRQIAEAVSYGVFKMNIDTDIQFAFAEGVGGYVLENPEAFRHQISPSTGKPLKKVYDPRKWLRVGENSIVSRLDQTFADLGATGRTVASS

SEQ ID NO: 2 - DNA, 921 bp, Gluconacetobacter xylinus
  misc_feature (1)..(921): DSM2325 M9
atgacactgacaccgcgggtcaaggcaatccttgaccactacgaaagtgacacgccgggcaccaaggccaatctctaccggctcatgaacaccggcaagctcgcgggtaccggcaagctggtgatcctgccggttgaccagggcttcgagcacgggccaggccgctcgtttgcccccaacccgcccgcctatgacccgcattatcactactcgctggccatcgaggcggggctgaacgcattcgcagccccgctgggcatgcttgaggccggggccggcacgtttgccggccagatccccaccattctcaaatgcaacagttccaacagcctgaccacgcagaagaaccaggccgtgaccggtacggtcgccgatgcgctgcggctgggctgctctgccatcgggtttaccatctaccccgccagcgactaccagttccaccagatggagcaactgcgcgagatggcacgcgaggccaagaatgcagggcttgccgtggtggtgtggagctacccgcgtggcccgatgctcgacaaggcgggcgagacggccatcgacatctgcgcctatgccgcccatatcgccgccgaactcggtgcccacatcatcaaggtcaagcccccgaccgaggatctgtgcctgcccgcggccaagaaggtctatatcgatgagaaggtcgatatcgccaccctgcccgcgcgaatccaccatgtggtgcagtcggcctttgcgggccgccgcatagtgatcttctcgggtggcgagcacaccaccaccgagcacctgctcgacaccattcgcggcatccatcagggtggcgggttcggttcgatcatcgggcgcaacaccttccagcgcccacgggccgaagccctgaagctgcttggtgacattaccgacatcttcctccagaaggtctga

SEQ ID NO: 3 - DNA, 1359 bp, Komagataeibacter xylinus
  misc_feature (1)..(1359): DSM2325 M9
atggtaaaaacaagaaagctgtttggcactgatggcattcggggcatggccaaccgctttcccatgacggtggaagtcgcgcagaagctgggccaggccgcgggcctgcgcttcatacagggcacgcaccgccatagcgtgctgctgggcaaggatacgcgcctgtcgggctatatgatcgaatgcgcgctggtgtcgggcttcctttccgccggaatggacgtgacgctggtggggccgatgcccaccccggccattgccatgctcacccgttccctgcgcgccgatctgggcgtcatgatctcggcgtcgcacaatccgtatggcgataacggcatcaagctgttcggccctgacgggttcaagctctccgatgaaacggaagcgggtattgaagcggcaatgagcgaggacctgacccatatgctcgccgcccccgaccagatcggccgggcctcgcgccttaatgacgcggcgggccggtacgtggaaagcgccaagtcctccttcccccgccgcctgcggcttgacgggctgcgcatcgtaatcgactgcgccaacggggcggcctatcgcgtggcgcccacggcattgtgggaactcggtgcggaagtggtgcgcataggctgcgaccctgatggcatcaacatcaatgaaggctgcggctccacccgccccgaggccctgtgtgctgccgtgcagcgccaccgggccgatatcggcatcgccctcgatggcgatgccgaccgcgtgctgatttctgatgaaaagggccgcctgatcgatggcgaccagatcctggcgctgatctcgcattcatgggcgcggcaggggcggctgtcggggcggcatatcgtggccaccgtcatgtccaacatggggcttgagcgctatctcgagacacaggggctggaactggtgcgcacggcggtgggcgatcgctacgtggtggaaaaaatgcgcgagcttggcgccaatatcggtggcgagcagtcagggcatatggtgctgtcggatttcgccaccacgggcgacgggctggtggcagccctgcaggtactggctgaagtggtggagtccggtcgccctgcaagcgaggtgtgccgcatgttcaagccctacccgcaactgctgcgcaacgtgcgctttgccgggcgcagcccgttgcatgacccgcaggtgcatgacgcgcgcaaggcggcggaaaagcggctgggcgcgcgcgggcgactcgtgttgcgtgaaagcggcaccgaaccgctggtgcgcgtcatggcggaagccgaggacgaagcgctggtcaatgcggtggtcgatgacatgtgcgaggcgattaccgccatccagatggcgggctga

SEQ ID NO: 4 - protein, 452 aa, Komagataeibacter xylinus
  misc_feature (1)..(452): DSM2325 M9
MVKTRKLFGTDGIRGMANRFPMTVEVAQKLGQAAGLRFIQGTHRHSVLLGKDTRLSGYMIECALVSGFLSAGMDVTLVGPMPTPAIAMLTRSLRADLGVMISASHNPYGDNGIKLFGPDGFKLSDETEAGIEAAMSEDLTHMLAAPDQIGRASRLNDAAGRYVESAKSSFPRRLRLDGLRIVIDCANGAAYRVAPTALWELGAEVVRIGCDPDGININEGCGSTRPEALCAAVQRHRADIGIALDGDADRVLISDEKGRLIDGDQILALISHSWARQGRLSGRHIVATVMSNMGLERYLETQGLELVRTAVGDRYVVEKMRELGANIGGEQSGHMVLSDFATTGDGLVAALQVLAEVVESGRPASEVCRMFKPYPQLLRNVRFAGRSPLHDPQVHDARKAAEKRLGARGRLVLRESGTEPLVRVMAEAEDEALVNAVVDDMCEAITAIQMAG

SEQ ID NO: 5 - DNA, 900 bp, Komagataeibacter xylinus
  misc_feature (1)..(900): DSM2325 M9
atgagcgaacacggtagcgcaaagccgaccaagggcattcttctggctggcgggtcgggcacgcgcctgcaccccatgacactggcagtcagcaagcagttgctgccggtctatgacaagccgatgatcttctacccgctttccacgctcatgctggcggggatacgcgatatcatgatcatttccaccccggccgacctgccgctgttccgcaggctgctcggcgatggggcggatatgggtgttaccttcacctaccgcgagcagcccgcgcccgatggtattgcccaggcttttgtcattgccgatgactggctggatgattcgccgtgcgggcttattctgggtgataacctgatctttgccgaccatctgggcaagcagatgcgtgcagccgccacccggccaagcggggccaccgtttttgcctatcaggtgcgtgaccccgagcgttatggcgtggtaagttttggcgaggacgggcatgcaatcgatattgttgaaaaacccaccgaacccaagtcaaactgggcagtaacggggctgtatttttatgatggtcgcgtgcgtgaatatgcgcgcagcctcaggccctcgccgcgtggcgaactggaaattaccgacctgaaccgcctttacctgcagtcggatgaactgcatgtgcagcgccttggccgcggctgtgcgtggcttgatgccggcatgcccgacagcctgatgcaggccgggcagttcgtgcagaccatccagtcccggcaggggctgctcgttggctcgccgcatgaggtggccttccgcatggggttcattgatgccgcggggcttgaagcctatgccaggcgcatgatcaagaccgaactgggccaggcgctcatggccattgcccatggcgagggataa

SEQ ID NO: 6 - protein, 299 aa, Komagataeibacter xylinus
  misc_feature (1)..(299): DSM2325 M9
MSEHGSAKPTKGILLAGGSGTRLHPMTLAVSKQLLPVYDKPMIFYPLSTLMLAGIRDIMIISTPADLPLFRRLLGDGADMGVTFTYREQPAPDGIAQAFVIADDWLDDSPCGLILGDNLIFADHLGKQMRAAATRPSGATVFAYQVRDPERYGVVSFGEDGHAIDIVEKPTEPKSNWAVTGLYFYDGRVREYARSLRPSPRGELEITDLNRLYLQSDELHVQRLGRGCAWLDAGMPDSLMQAGQFVQTIQSRQGLLVGSPHEVAFRMGFIDAAGLEAYARRMIKTELGQALMAIAHGEG

SEQ ID NO: 7 - DNA, 33 bp, Artificial Sequence
  Synthetic primer F1-F
cggcgtagaggatcaggagcttatcgactgcac

SEQ ID NO: 8 - DNA, 28 bp, Artificial Sequence
  Synthetic primer F1-R
ccggcgtagagaatccacaggacgggtg

SEQ ID NO: 9 - DNA, 27 bp, Artificial Sequence
  Synthetic primer F2-F
ctgtggattctctacgccggacgcatc

SEQ ID NO: 10 - DNA, 29 bp, Artificial Sequence
  Synthetic primer F2-R
aagggcatcggtcgtcgctctcccttatg

SEQ ID NO: 11 - DNA, 3128 bp, Artificial Sequence
  Synthetic pTSa-EX1 vector
gaattcagccagcaagacagcgatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatcacctgtaagtcggacgcgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaaaggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacatagatctcccgggtaccgagctctctagaaagaaggagggacgagctattgatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttttttaaggcagttattggtgcccttaaacgcctggttgctacgcctgaataagtgataataagcggatgaatggcagaaattc

SEQ ID NO: 12 - DNA, 32 bp, Artificial Sequence
  Synthetic primer GX1979_F
tcctctagagtcgacatgacactgacaccgcg

SEQ ID NO: 13 - DNA, 37 bp, Artificial Sequence
  Synthetic primer GX1979_R
tgcctgcaggtcgactcagaccttctggaggaagatg

SEQ ID NO: 14 - DNA, 40 bp, Artificial Sequence
  Synthetic primer gx1215-F
tcctctagagtcgacatggtaaaaacaagaaagctgtttg

SEQ ID NO: 15 - DNA, 34 bp, Artificial Sequence
  Synthetic primer gx1215-R
tgcctgcaggtcgactcagcccgccatctggatg

SEQ ID NO: 16 - DNA, 33 bp, Artificial Sequence
  Synthetic primer gx2556-F
tcctctagagtcgacatgagcgaacacggtagc

SEQ ID NO: 17 - DNA, 33 bp, Artificial Sequence
  primer gx2556-R
tgcctgcaggtcgacttatccctcgccatgggc

SEQ ID NO: 18 - DNA, 2166 bp, Komagataeibacter xylinus
atgatctggcgcattttaaaatctcccctcgtctccggcccgttattcgccatcctcctggcagtggtctgcctgacctacctctcccccgaccaccagtttttcgtcgcgatagggggcgcgatcctgttctttctggttcgccgacatgatgaacgctggtcgcgctgttttctcatggtgctgtccatcgtggtatccgggcgctatctggtgtggcgctttacctccacgcttgatctcgatggcgtgttgcagacagttctagtcctggcgctggcgatcggcgaaatctataccaccttccgggtgggctttacgtatttccagttggcctggcccctgcggcggcagatccacccgctgccggaagatgaaggcagttggccggtcattgatgtctatgtgccaacctataacgaggacatggcgatcgtccgcaccacggtgctgggctgcctggccatggactggccggcagacaagctgaatgtctatatccttgatgacgggcggcggcgctcgttccgtgattttgccgcgcaggtcggtgctggctacatcaatcgcgcggacagtacccacgccaaggcgggcaacctcaaccatgccatcaaggcgacaacgggcgacctgatcgcgatctttgactgtgaccatgtgcccgtgcggggtttcctcaaaaagaccgtggggtggatgatcgccgaccccaacctcgcgctattgcagaccccgcatcacttctattcccccgatccgttccgtcgcaacatgagccggggcatgcaggtgccgcccgagagcaacctgttctatgggcttttgcaagatggcaatgatttctggaacgccaccttcttctgcgggtcgtgcgccctgctgcggcgcgaggccattgaagcgatcaatggctttgccgtcgagaccgtgacggaagatgcccacaccgccctgcgcatgcagcgcaaggggtggggcacggcctatctgcgcgagccgctggccgcggggctcgaaaccgaaaccctcctgcttcaggtcgggcagcgcgtgcgctgggcgcgcggcatgatccagatgctgcggctcgacaaccccatgctcggccgtggcctgcgcctcacgcagcgtatctgctacatggcggcgacgacgaactacttcttcgccatgccgcgcatcatgttcctcatggcgccgctggcctacctgttcctgggcgtgaccatgatcgcggcctcgccttatgaacttgcggtctatgccctgccgcacctgtttcataccaccatgaccatgtcgcgcctgcaggggcggtggcgctattcgttctggagcgagatctacgaatccatgctggccccctttctggtgcgcatgacgttcatcaccctgcttgcgccgcacaagggcaagttcaacgtgaccgacaagggcggcctgctgcaccgcgagtattttgaatggcgcgcggcctaccccggcgtgatcatggccgtggtgctggcggtgggactggtgagcggcatctgggccgcgattgccccttatcatgaaacgctcgtcttccgcgccatggcggtcaactcggtctgggtgctgttcagcctgatcatcgtgcttggtggtgtggccgccgcgcgcgaaacccgccagcgccgccgtaaccaccgcgttgcggccagcattcccctgaccatgttcacgggtgatacgcaggtcaccgcctgtcgcacgctggatgtgtcgatggggggctgccagcttgacctgtcgcccacactgccccttgccgtgggggatgaactgcgcctgcacgccaccctggcctccggcccgatcacgctccgcgccaccctcatcgaccggcatgagggccgtgcccatgtggcgtggatcatgcccgacctcgcggccgagaagcaggtcgtggccctggtgtttggccgtgatgatgcctggtcccagtggtccgacttcccgcctgacaggccgcttcacagtctttacatgctgcttgccagcatctgcgcgctgttccgcccctatccgcgcgggcagtcggatgcgccgccaccgcccgcgccgcctcccccgatcgcagaggaaaaactgccggcacggcatctggttataccaaccgttgattgctataacctatga

SEQ ID NO: 19 - protein, 721 aa, Komagataeibacter xylinus
MIWRILKSPLVSGPLFAILLAVVCLTYLSPDHQFFVAIGGAILFFLVRRHDERWSRCFLMVLSIVVSGRYLVWRFTSTLDLDGVLQTVLVLALAIGEIYTTFRVGFTYFQLAWPLRRQIHPLPEDEGSWPVIDVYVPTYNEDMAIVRTTVLGCLAMDWPADKLNVYILDDGRRRSFRDFAAQVGAGYINRADSTHAKAGNLNHAIKATTGDLIAIFDCDHVPVRGFLKKTVGWMIADPNLALLQTPHHFYSPDPFRRNMSRGMQVPPESNLFYGLLQDGNDFWNATFFCGSCALLRREAIEAINGFAVETVTEDAHTALRMQRKGWGTAYLREPLAAGLETETLLLQVGQRVRWARGMIQMLRLDNPMLGRGLRLTQRICYMAATTNYFFAMPRIMFLMAPLAYLFLGVTMIAASPYELAVYALPHLFHTTMTMSRLQGRWRYSFWSEIYESMLAPFLVRMTFITLLAPHKGKFNVTDKGGLLHREYFEWRAAYPGVIMAVVLAVGLVSGIWAAIAPYHETLVFRAMAVNSVWVLFSLIIVLGGVAAARETRQRRRNHRVAASIPLTMFTGDTQVTACRTLDVSMGGCQLDLSPTLPLAVGDELRLHATLASGPITLRATLIDRHEGRAHVAWIMPDLAAEKQVVALVFGRDDAWSQWSDFPPDRPLHSLYMLLASICALFRPYPRGQSDAPPPPAPPPPIAEEKLPARHLVIPTVDCYNL